(19) United States
(12) Patent Application Publication
(10) Pub. No.: US 2020/0187784 A1
(43) Pub. Date: Jun. 18, 2020

(54) PHOTOACOUSTIC IMAGE GENERATION APPARATUS AND IMAGE ACQUISITION METHOD

(71) Applicant: FUJIFILM CORPORATION, Tokyo (JP)

(72) Inventor: Atsushi HASHIMOTO, Kanagawa (JP)

(21) Appl. No.: 16/802,568

(22) Filed: Feb. 27, 2020

Related U.S. Application Data
(63) Continuation of application No. PCT/JP2018/030837, filed on Aug. 21, 2018.

(30) Foreign Application Priority Data
Aug. 29, 2017 (JP) 2017-164587

(51) Int. Cl. A61B 5/00

(52) U.S. Cl. CPC A61B 5/0095; A61B 2576/00

(57) ABSTRACT

A control unit of a photoacoustic image generation apparatus performs control to adjust an excitation light generation condition based on a pulse width, the number of a plurality of pulses, and a pulse repetition period of excitation light generated in a light source based on reception frequency characteristics of an acoustic wave detection means with respect to the light source.

### CROSS-REFERENCE TO RELATED APPLICATIONS

This application is a continuation application of International Application No. PCT/JP2018/030837, filed Aug. 21, 2018, the disclosure of which is incorporated herein by reference in its entirety. Further, this application claims priority from Japanese Patent Application No.2017-164587, filed Aug. 29, 2017, the disclosure of which is incorporated herein by reference in its entirety.

### BACKGROUND OF THE INVENTION

### 1. Field of the Invention

The present invention relates to a photoacoustic image generation apparatus that generates a photoacoustic image based on a signal obtained by detecting a photoacoustic wave generated from the inside of a subject by receiving excitation light emitted from a light source toward the subject and an image acquisition method in a photoacoustic image generation apparatus.

### 2. Description of the Related Art

In recent years, a noninvasive measurement method using a photoacoustic effect has attracted attention. In this measurement method, pulsed light having a certain appropriate wavelength (for example, wavelength range of visible light, near-infrared light, or mid-infrared light) is emitted toward a subject, a photoacoustic wave which is an elastic wave generated as a result of absorbing energy of the pulsed light by an absorbing substance in the subject is detected, and the concentration of the absorbing substance is quantitatively measured. Examples of the absorbing substance in the subject include a blood vessel, glucose, and hemoglobin contained in blood. In addition, a technique of detecting such a photoacoustic wave and generating a photoacoustic image based on the detection signal is referred to as photoacoustic imaging (PAI) or photoacoustic tomography (PAT).

For example, JP2016-047232A and JP2016-047077A disclose apparatuses that perform photoacoustic imaging to generate a photoacoustic image. This type of photoacoustic image generation apparatus is often formed to generate also a so-called reflected ultrasound image.

In general, an apparatus that generates a reflected ultrasound image generates a tomographic image or the like of the inside of a subject based on a signal obtained by detecting a reflected acoustic wave that an acoustic wave (mostly an ultrasonic wave) emitted toward the subject is reflected in the subject.

On the other hand, a photoacoustic image generation apparatus generally emits excitation light such as laser light toward a subject to generate a photoacoustic image indicating an internal tissue or the like of the subject based on a signal obtained by detecting a photoacoustic wave generated from a site that absorbs the excitation light.

### SUMMARY OF THE INVENTION

In the photoacoustic imaging as described above, there are three methods for improving a visible depth in the photoacoustic image: (1) Increase energy of a photoacoustic wave generated in a subject, (2) Improve reception efficiency of an ultrasound probe that detects the generated photoacoustic wave, and (3) Reduce a background noise of the image.

In order to increase the energy of the photoacoustic wave generated in the subject in (1), it is conceivable to increase energy of excitation light irradiated in the subject. However, there is a limit to increase peak energy of one pulse of the excitation light due to a restriction on hardware of a light source. In order to reduce the background noise of the image in (3), it is conceivable that one image is formed using a plurality of photoacoustic images or reception data of one line is formed using reception data of a plurality of waves. However, there is a problem that a frame rate is lowered in this case.

For this reason, it is preferable to improve the reception efficiency of the ultrasound probe that detects the generated photoacoustic wave in (2) in order to improve the visible depth in the photoacoustic image.

In this regard, JP2016-047232A discloses that a pulse width of excitation light for generating a photoacoustic wave is optimized according to an ultrasound probe such that the ultrasound probe can efficiently receive the photoacoustic wave. However, the method of JP2016-047232A is insufficient in efficiency due to, for example, generation of many acoustic waves having frequency components that do not contribute to imaging.

Further, JP2016-047077A discloses that a pulse width of excitation light and the number of pulses thereof are determined according to reception frequency characteristics of an ultrasound probe such that the ultrasound probe can efficiently receive a photoacoustic wave. Furthermore, JP2016-047077A discloses that a pulse repetition period is changed while the pulse width is maintained constant after the pulse width of the excitation light and the number of pulses thereof are determined according to the reception frequency characteristics of the ultrasound probe so as to be able to improve a resolution. However, in a case where the pulse repetition period is changed after the pulse width of the excitation light is determined, a band of the generated photoacoustic wave is changed. Therefore, there is a problem that the reception frequency characteristics of the ultrasound probe do not match and the reception efficiency of the ultrasound probe is reduced.

In view of the circumstances described above, an object of the invention is to provide a photoacoustic image generation apparatus that improves a visible depth in a photoacoustic image and an image acquisition method in a photoacoustic image generation apparatus.

In a photoacoustic image generation apparatus according to an embodiment of the invention comprising a photoacoustic image generation unit that generates a photoacoustic image based on a signal obtained by detecting a photoacoustic wave, using an acoustic wave detection means, generated from an inside of a subject by receiving excitation light emitted from a light source toward the subject, the photoacoustic image generation apparatus comprises a control unit that performs control to adjust an excitation light generation condition based on a pulse width, the number of a plurality of pulses, and a pulse repetition period of the excitation light generated in the light source based on reception frequency characteristics of the acoustic wave detection means with respect to the light source.

In the photoacoustic image generation apparatus according to the embodiment of the invention, the control unit may perform control to adjust the excitation light generation condition such that frequency characteristics of the photoacoustic wave detected by the acoustic wave detection means are close to the reception frequency characteristics of the acoustic wave detection means.

The control unit may store a plurality of excitation light generation conditions having different frequency characteristics of photoacoustic waves generated in the subject and control the light source based on the excitation light generation condition selected from among the plurality of stored excitation light generation conditions.

In this case, the control unit may store a plurality of excitation light generation conditions for each type having different reception frequency characteristics of the acoustic wave detection means and control the light source based on the excitation light generation condition selected by a user from among the plurality of stored excitation light generation conditions.

In addition, the control unit may adjust the excitation light generation condition based on an image depth of the photoacoustic image.

In addition, the control unit may adjust the excitation light generation condition based on a focal depth of the photoacoustic image.

In addition, the photoacoustic image generation unit may perform correction processing on the photoacoustic image based on the excitation light generation condition.

An image acquisition method according to an embodiment of the invention is an image acquisition method in a photoacoustic image generation apparatus comprising a photoacoustic image generation unit that generates a photoacoustic image based on a signal obtained by detecting a photoacoustic wave, using an acoustic wave detection means, generated from an inside of a subject by receiving excitation light emitted from a light source toward the subject. The image acquisition method performs control to adjust an excitation light generation condition based on a pulse width, the number of a plurality of pulses, and a pulse repetition period of the excitation light generated in the light source based on reception frequency characteristics of the acoustic wave detection means with respect to the light source.

In the image acquisition method according to the embodiment of the invention, the excitation light generation condition may be controlled to be adjusted such that frequency characteristics of the photoacoustic wave detected by the acoustic wave detection means are close to the reception frequency characteristics of the acoustic wave detection means.

In addition, a plurality of excitation light generation conditions having different frequency characteristics of photoacoustic waves generated in the subject may be stored and the light source may be controlled based on the excitation light generation condition selected from among the plurality of stored excitation light generation conditions.

In this case, a plurality of excitation light generation conditions for each type having different reception frequency characteristics of the acoustic wave detection means may be stored and the light source may be controlled based on the excitation light generation condition selected by a user from among the plurality of stored excitation light generation conditions.

In addition, the excitation light generation condition may be adjusted based on an image depth of the photoacoustic image.

In addition, the excitation light generation condition may be adjusted based on a focal depth of the photoacoustic image.

In addition, correction processing may be performed on the photoacoustic image based on the excitation light generation condition.

With the photoacoustic image generation apparatus and the image acquisition method according to the embodiment of the invention, the control to adjust the excitation light generation condition based on the pulse width, the number of a plurality of pulses, and the pulse repetition period of excitation light generated in the light source is performed based on the reception frequency characteristics of the acoustic wave detection means with respect to the light source. Therefore, it is possible to improve the reception efficiency of the photoacoustic wave in the acoustic wave detection means and thus improve visible depth in the photoacoustic image.

### BRIEF DESCRIPTION OF THE DRAWINGS

FIG. 1 is a block diagram illustrating the schematic configuration of a photoacoustic image generation apparatus according to a first embodiment of the invention.

FIG. 2 is a graph illustrating waveforms of excitation light.

FIG. 3 is a graph illustrating waveforms of photoacoustic waves.

FIG. 4 is a graph illustrating spectra of photoacoustic waves.

FIG. 5 is a graph illustrating spectra of photoacoustic waves.

FIG. 6 is a graph illustrating waveforms of excitation light.

FIG. 7 is a graph illustrating spectra of photoacoustic waves.

FIG. 8 is a graph illustrating waveforms of excitation light.

FIG. 9 is a graph illustrating spectra of photoacoustic waves.

FIG. 10 is a graph illustrating a waveform of excitation light.

FIG. 11 is a graph illustrating spectra of photoacoustic waves.

FIG. 12 is a graph illustrating a waveform of excitation light.

FIG. 13 is a graph illustrating spectra of photoacoustic waves.

FIG. 14 is a graph illustrating waveforms of excitation light.

FIG. 15 is a graph illustrating spectra of photoacoustic waves.

FIG. 16 is a graph illustrating spectra of photoacoustic waves.

FIG. 17 is a graph illustrating spectra of photoacoustic waves.

### DESCRIPTION OF THE PREFERRED EMBODIMENTS

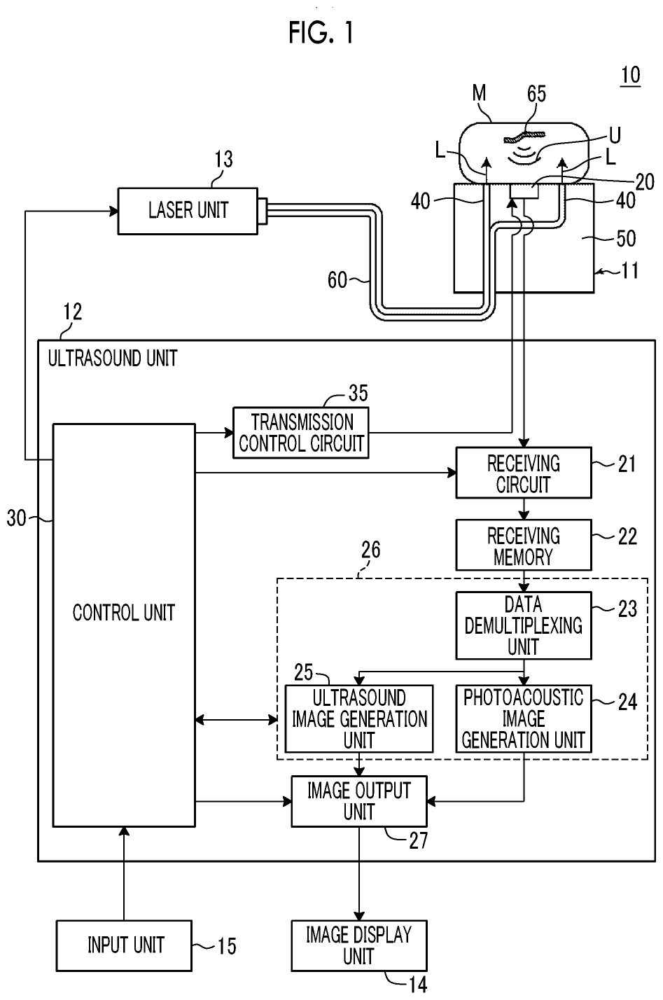

Hereinafter, embodiments of the invention will be described in detail with reference to drawings. FIG. 1 is a schematic diagram illustrating the entire configuration of a photoacoustic image generation apparatus 10 according to a first embodiment of the invention. FIG. 1 schematically illustrates a shape of an ultrasound probe (hereinafter simply referred to as a probe) 11. The photoacoustic image generation apparatus 10 of this example has a function of generating a photoacoustic image based on a photoacoustic wave detection signal and comprises a probe 11, an ultrasound unit 12, a laser unit 13, an image display unit 14, an input unit 15, and the like as schematically illustrated in FIG. 1. Hereinafter, these components will be sequentially described.

The probe 11 as an acoustic wave detection means has, for example, a function of emitting excitation light and an ultrasonic wave toward a subject M which is a living body and a function of detecting an acoustic wave U propagating in the subject M. That is, the probe 11 can emit (transmit) an ultrasonic wave (acoustic wave) to the subject M and detect (receive) a reflected ultrasonic wave (reflected acoustic wave) reflected back from the subject M.

In this specification, the “acoustic wave” is a term including an ultrasonic wave and a photoacoustic wave. Here, the “ultrasonic wave” means an elastic wave transmitted by the probe 11 and a reflected wave (reflected ultrasonic wave) thereof, and the “photoacoustic wave” means an elastic wave emitted by an absorber 65 absorbing the excitation light. In addition, the acoustic wave emitted from the probe 11 is not limited to the ultrasonic wave, and an acoustic wave having an audible frequency may be used as long as an appropriate frequency is selected according to a subject to be examined and a measurement condition. Examples of the absorber 65 in the subject M include a blood vessel, glucose, and hemoglobin contained in blood, and a metal member.

In general, probes 11 corresponding to sector scanning, linear scanning, convex scanning, and the like are prepared, and an appropriate probe is selected and used according to a site to be imaged. In addition, an optical fiber 60 is connected to the probe 11 as a connection portion that guides laser light L which is the excitation light emitted from a laser unit 13 described below to a light emission portion 40.

The probe 11 comprises a vibrator array 20 which is an acoustic wave detector, a total of two light emission portions 40 that are disposed on both sides of the vibrator array 20 for one on each side with the vibrator array 20 interposed therebetween, and a housing 50 in which the vibrator array 20, the two light emission portions 40, and the like are accommodated.

In the embodiment, the vibrator array 20 also functions as an ultrasound transmission element. The vibrator array 20 is connected to a transmission control circuit 35 of the ultrasonic wave, a receiving circuit 21 thereof, and the like through wiring not illustrated.

The vibrator array 20 is a vibrator array in which a plurality of acoustic wave vibrators (ultrasound vibrators) which are electroacoustic transducers are arranged to be parallel in a one-dimensional direction. The acoustic wave vibrator is a piezoelectric element made of, for example, piezoelectric ceramics. The acoustic wave vibrator may be a piezoelectric element made of a polymer film such as polyvinylidene fluoride (PVDF). The acoustic wave vibrator has a function of converting the received acoustic wave U into an electric signal. The vibrator array 20 may include an acoustic lens.

As described above, the vibrator array 20 according to the embodiment is the vibrator array in which the plurality of acoustic wave vibrators are arranged to be parallel one-dimensionally, but may be a vibrator array in which a plurality of acoustic wave vibrators are arranged to be parallel two-dimensionally.

The acoustic wave vibrator also has a function of transmitting the ultrasonic wave as described above. That is, in a case where an alternating voltage is applied to the acoustic wave vibrator, the acoustic wave vibrator generates an ultrasonic wave having a frequency corresponding to a frequency of the alternating voltage. The transmission and reception of the ultrasonic wave may be separated from each other. That is, for example, the ultrasonic wave may be transmitted from a position different from the probe 11, and the probe 11 may receive a reflected ultrasonic wave with respect to the transmitted ultrasonic wave.

The light emission portion 40 emits the laser light L guided by the optical fiber 60 toward the subject M. In the embodiment, the light emission portion 40 is formed by a tip portion of the optical fiber 60, that is, an end portion far from the laser unit 13 which is a light source of the excitation light. As illustrated in FIG. 1, in the embodiment, two light emission portions 40 are disposed on both sides of the vibrator array 20, for example, in an elevation direction with the vibrator array 20 interposed therebetween. The elevation direction is a direction that is perpendicular to an arrangement direction of the acoustic wave vibrators and parallel to a detection surface of the vibrator array 20 in a case where the plurality of acoustic wave vibrators are arranged to be parallel one-dimensionally.

The light emission portion may be formed by a light guide plate and a diffusion plate that are optically coupled to the tip of the optical fiber 60. Such a light guide plate can be made of, for example, an acrylic plate or a quartz plate. A lens diffusion plate in which microlenses are randomly disposed on a substrate can be used as the diffusion plate. Further, for example, a quartz plate in which diffusion fine particles are distributed or the like can be used. Furthermore, a holographic diffusion plate or an engineering diffusion plate may be used as the lens diffusion plate.

The laser unit 13 as the light source has, for example, a flash lamp excitation Q-switch solid laser such as a Q-switch alexandrite laser and generates the laser light L as the excitation light. The laser unit 13 receives a trigger signal from a control unit 30 of the ultrasound unit 12 and outputs the laser light L.

A wavelength of the laser light L is appropriately selected according to light absorption characteristics of the absorber 65 in the subject M to be measured. For example, in a case where a measurement target is hemoglobin in a living body, that is, in a case where a blood vessel is imaged, it is generally preferable that the wavelength of the laser light L is a wavelength belonging to a near-infrared wavelength range. The near-infrared wavelength range means a wavelength range of about 700 to 2500 nm (nanometer). However, the wavelength of the laser light L is naturally not limited thereto. The laser light L may be a single wavelength or may include a plurality of wavelengths such as 750 nm (nanometer) and 800 nm (nanometer). In a case where the laser light L includes a plurality of wavelengths, the light beams having these wavelengths may be emitted at the same time or may be emitted alternately while being switched.

The laser unit 13 may be formed by using a yttrium aluminum garnet (YAG)-second harmonic generation (SHG)-optical parametric oscillation (OPO) laser, a titanium-sapphire laser, or the like capable of outputting laser light having the near-infrared wavelength range similarly in addition to the alexandrite laser described above.

In addition, the laser unit 13 may also be formed by using a laser diode (LD) or a light emitting diode (LED). The light source may be the LD or the LED having a lower output instead of the solid laser having a higher output since the invention improves the reception efficiency of the photoacoustic wave. In addition, the LD or the LED is generally preferable as compared with the solid laser in order to generate excitation light having a predetermined waveform as described below.

The optical fiber 60 guides the laser light L emitted from the laser unit 13 to the two light emission portions 40. The optical fiber 60 is not particularly limited, and a known fiber such as a quartz fiber may be used. For example, one thick optical fiber may be used, or a bundle fiber in which a plurality of optical fibers are bundled may be used. In a case where the bundle fiber is used as an example, the bundle fiber is disposed such that the laser light L is incident from a light incident end surface of a bundled fiber portion, and each tip portion of fiber portions branched into two of the bundle fiber forms the light emission portion 40 as described above.

The ultrasound unit 12 includes the receiving circuit 21, a receiving memory 22, an image generation unit 26, an image output unit 27, the control unit 30, and the transmission control circuit 35. The image generation unit 26 is formed of a data demultiplexing unit 23, a photoacoustic image generation unit 24, and an ultrasound image generation unit 25. The control unit 30 comprises a function of, for example, controlling adjustment of an excitation light generation condition based on a pulse width, the number of a plurality of pulses, and a pulse repetition period of the laser light L generated by the laser unit 13 based on reception frequency characteristics of the probe 11 with respect to the laser unit 13 as the light source. The ultrasound unit 12 typically has, for example, a processor, a memory, and a bus. In the ultrasound unit 12, a program relating to photoacoustic image generation processing, ultrasound image generation processing, control processing with respect to the laser unit 13, and the like is incorporated in a memory not illustrated. The function of each unit is realized by the program being operated by the control unit 30 formed by a processor. That is, each of these units is formed by the memory into which the program is incorporated and the processor.

A hardware configuration of the ultrasound unit 12 is not particularly limited and can be realized by combining a plurality of integrated circuits (ICs), a processor, an application specific integrated circuit (ASIC), a field-programmable gate array (FPGA), a memory, and the like as appropriate.

The receiving circuit 21 receives a detection signal output from the ultrasound probe 11 and stores the received detection signal in the receiving memory 22. The receiving circuit 21 typically includes a low-noise amplifier, a variable-gain amplifier, a low pass filter, and an analog to digital converter (AD). The probe 11 is amplified by the low-noise amplifier, is subjected to gain adjustment corresponding to a depth by the variable-gain amplifier, is converted into a digital signal by the AD converter after high-frequency components of the detection signal is cut by the low pass filter, and then is stored in the receiving memory 22. The receiving circuit 21 is formed by, for example, one IC.

The probe 11 outputs a detection signal of the photoacoustic wave and a detection signal of the reflected ultrasonic wave. The receiving memory 22 stores the AD-converted detection signals (sampling data) of the photoacoustic wave and the reflected ultrasonic wave. The data demultiplexing unit 23 reads out the detection signal of the photoacoustic wave from the receiving memory 22 and transmits the detection signal to the photoacoustic image generation unit 24. The data demultiplexing unit 23 reads out the detection signal of the reflected ultrasonic wave from the receiving memory 22 and transmits the detection signal to the ultrasound image generation unit 25.

The photoacoustic image generation unit 24 generates a photoacoustic image based on the detection signal of the photoacoustic wave detected by the probe 11. The photoacoustic image generation processing includes, for example, image reconfiguration such as phase matching addition, detection, and logarithmic conversion. The ultrasound image generation unit 25 generates an ultrasound image based on the detection signal of the reflected ultrasonic wave detected by the probe 11. The ultrasound image generation processing also includes image reconfiguration such as phase matching addition, detection, logarithmic conversion, and the like. The image output unit 27 outputs the photoacoustic image and/or the ultrasound image on the image display unit 14 such as a display apparatus.

The control unit 30 controls each unit in the ultrasound unit 12. In a case where a photoacoustic image is acquired, the control unit 30 transmits the trigger signal to the laser unit 13 based on the excitation light generation condition described below to cause the laser unit 13 to emit laser light L. In addition, the control unit 30 transmits a sampling trigger signal to the receiving circuit 21 to control a sampling start timing of the photoacoustic wave or the like with the emission of the laser light L. The sampling data received by the receiving circuit 21 is stored in the receiving memory 22.

The photoacoustic image generation unit 24 receives the sampling data of the detection signal of the photoacoustic wave through the data demultiplexing unit 23 and performs detection at a predetermined detection frequency to generate the photoacoustic image. The photoacoustic image generated by the photoacoustic image generation unit 24 is input to the image output unit 27.

In a case in which an ultrasound image is acquired, the control unit 30 transmits an ultrasound transmission trigger signal for instructing the transmission control circuit 35 to transmit the ultrasonic wave. In a case where the ultrasound transmission trigger signal is received, the transmission control circuit 35 causes the probe 11 to transmit the ultrasonic wave. In a case in which the ultrasound image is acquired, the probe 11 performs a scanning, for example, while shifting a reception region of a group of piezoelectric elements line by line to detect the reflected ultrasonic wave by the control of the control unit 30. The control unit 30 transmits the sampling trigger signal to the receiving circuit 21 according to a transmission timing of the ultrasonic wave to start the sampling of the reflected ultrasonic wave. The sampling data received by the receiving circuit 21 is stored in the receiving memory 22.

The ultrasound image generation unit 25 receives the sampling data of the detection signal of the ultrasonic wave through the data demultiplexing unit 23 and performs the detection at a predetermined detection frequency to generate the ultrasound image. The ultrasound image generated by the ultrasound image generation unit 25 is input to the image output unit 27.

Here, a method of acquiring the photoacoustic image in the photoacoustic image generation apparatus 10 according to the embodiment will be described in detail. At the time of acquiring the photoacoustic image, the control unit 30 performs control with respect to the laser unit 13 (light source) to adjust the excitation light generation condition based on the pulse width, the number of a plurality of pulses, and the pulse repetition period of the laser light L (excitation light) generated by the laser unit 13 based on the reception frequency characteristics of the probe 11 (acoustic wave detection means). Here, a center frequency is considered as the frequency characteristics, but other frequency characteristics such as a peak frequency may be used.

The control unit 30 controls the adjustment of the excitation light generation condition based on the reception frequency characteristics of the probe 11 (acoustic wave detection means), that is, the center frequency in the sensitivity of the probe 11.

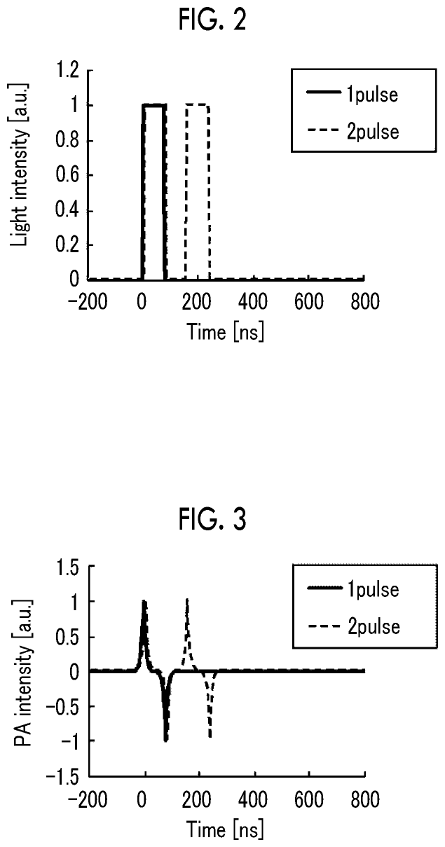

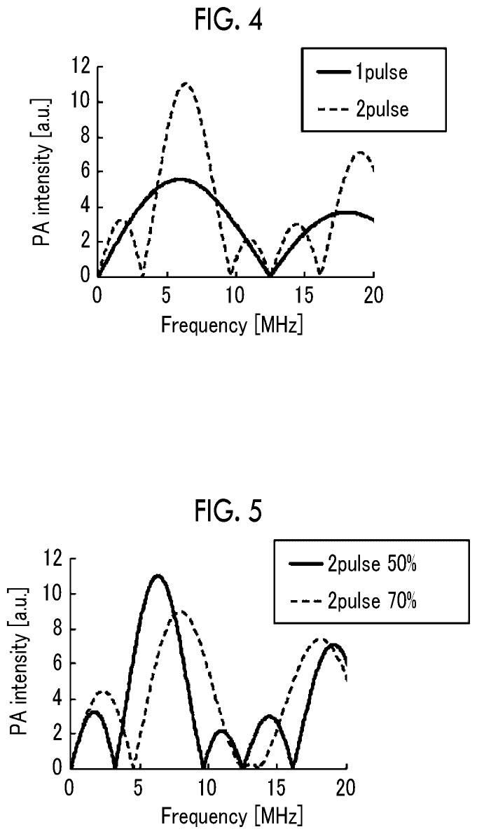

For example, a case is considered in which the center frequency in the sensitivity of the probe 11 is 6.5 MHz (megahertz). In this case, it is desirable that a center frequency of the photoacoustic wave generated in the subject M is near 6.5 MHz (megahertz). FIG. 2 is a graph illustrating waveforms of the excitation light, FIG. 3 is a graph illustrating waveforms of the photoacoustic waves, and FIG. 4 is a graph illustrating spectra of the photoacoustic waves. As illustrated in FIGS. 2 to 4, since it can be considered that the photoacoustic wave is generated at the intensity edge in the waveform of the laser light L (excitation light), in a case where the number of pulses of the laser light L is one, it is possible to generate a photoacoustic wave having the center frequency of 6.5 MHz in a case where the pulse width t_{LP} of the laser light L is set to 1/(2×6.5M)=77 ns (nanosecond).

In contrast, similarly as illustrated in FIGS. 2 to 4, in a case where the number of pulses of the laser light L is set to two or more, the pulse width t_{LP} of the laser light L is set to 1/(2×6.5M)=77 ns (nanosecond), and the pulse repetition period t_{LR} of the laser light L is set to 1/6.5M=154 ns (nanosecond), it is possible to generate a photoacoustic wave having the center frequency near 6.5 MHz (megahertz), larger peak spectrum intensity than that in the case where the number of pulses is one, and a narrower bandwidth than in the case where the number of pulses is one.

In general, since the reception frequency band of the probe 11 is a band having a width of 70% to 100% with respect to the center frequency, the frequency of the acoustic wave receivable by the probe having the center frequency of 6.5 MHz (megahertz) is considered to be about 3.2 to 9.8 MHz (megahertz). In a case where the number of pulses of the laser light L is one, the reception efficiency of the photoacoustic wave in the probe 11 is low due to the generation of many photoacoustic waves other than the frequency receivable by the probe 11 as illustrated in FIG. 4. On the other hand, in a case where the number of pulses of the laser light L is two, the reception efficiency of the photoacoustic wave in the probe 11 is high since most of the generated photoacoustic waves have frequencies receivable by the probe 11.

Here, it is considered to shorten the pulse repetition period (increase duty ratio) in order to improve a resolution. FIG. 5 is a graph illustrating spectra of the photoacoustic waves. In a case where the pulse repetition period t_{LR} of the laser light L is 110 ns (nanosecond) in order to set the duty ratio to 70%, the spectrum of the photoacoustic wave is changed as compared with a case where the ratio is 50% (pulse repetition period t_{LR}=154 ns (nanosecond) of laser light L) as illustrated in FIG. 5. For this reason, the reception efficiency of the photoacoustic wave in the probe 11 is lowered in the method of JP2016-047077A in which the pulse repetition period (duty ratio) is changed after the pulse width and the number of pulses of the laser light L are determined according to the reception frequency characteristics of the probe 11.

In contrast, in the embodiment, the excitation light generation condition is adjusted based on the pulse width, the number of a plurality of pulses, and the pulse repetition period of the laser light L (excitation light) such that the center frequency of the photoacoustic wave is close to the center frequency in the sensitivity of the probe 11.

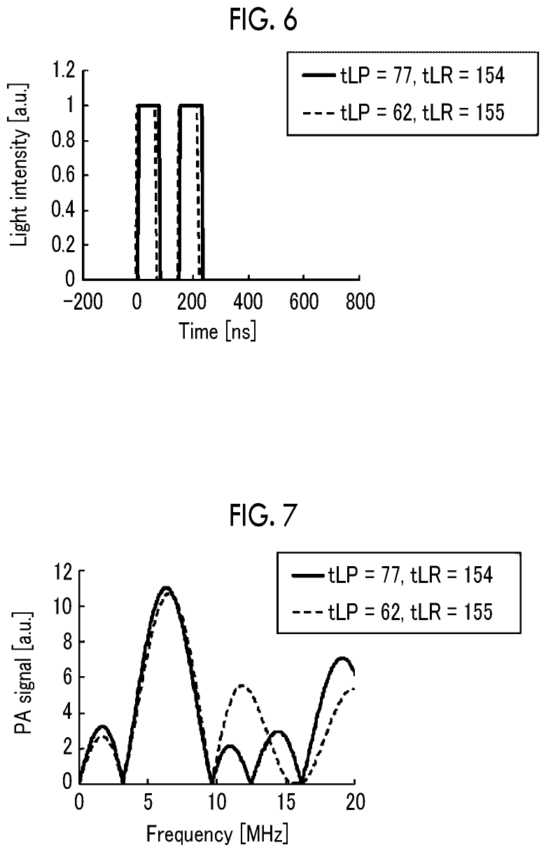

For example, FIG. 6 is a graph illustrating waveforms of the excitation light, FIG. 7 is a graph illustrating spectra of the photoacoustic waves. As illustrated in FIGS. 6 and 7, in a case where the number of pulses of the laser light L is set to two, the pulse width t_{LP} of the laser light L is set to 62 ns (nanosecond), and the pulse repetition period t_{LR} of the laser light L is set to 155 ns (nanosecond), it is possible to generate the photoacoustic wave having a center frequency near 6.5 MHz (megahertz) in the substantially same as the case where the number of pulses of the laser light L is set to two, the pulse width t_{LP} of the laser light L is set to 77 ns (nanosecond), and the pulse repetition period t_{LR} of the laser light L is set to 154 ns (nanosecond).

In a case where a laser diode (LD) or a light emitting diode (LED) is used as the laser unit 13 (light source), a method capable of generating a photoacoustic wave having the same degree of intensity as a long light emission time in a short light emission time is desirable in terms of low power consumption since a light emission time of the laser light L is proportional to power consumption. In addition, it is possible to expect a longer lifetime by shortening the light emission time since the lifetime of the light emitting device is considered to be caused by a total light emission time.

A center frequency Fc of the photoacoustic wave generated in a case where the subject M is irradiated with the laser light L is determined depending on both the pulse width t_{LP} of the laser light L and the pulse repetition period t_{LR} of the laser light L. Therefore, strictly speaking, it is desirable to calculate individually both the pulse width t_{LP} and the repetition period t_{LR} for center frequency Fc. However, Fc≈1/t_{LR} may be used as the first approximation since the contribution of the repetition period t_{LR} is generally larger.

In a case where t_{LP}≠t_{LR}/2, the generated photoacoustic wave includes more frequency components. Therefore, the bandwidth of the generated photoacoustic wave is generally wider than that in a case where t_{LP}=t_{LR}/2. It is also possible to adjust the bandwidth of the generated photoacoustic wave using this effect.

With the above control, it is possible to enhance the reception efficiency of the photoacoustic wave in the probe 11.

The number of pulses of the laser light L is not limited to two and may be three or more.

For example, in a case where the number of pulses of the laser light L increases, an object position in the photoacoustic image changes due to a change in the excitation light generation condition such as the object position in the photoacoustic image being shifted in a deep direction. For this reason, it is desirable that the photoacoustic image generation unit 24 corrects the object position in the photoacoustic image according to the excitation light generation condition.

The center frequency of the photoacoustic wave generated in the subject M and the center frequency in the sensitivity of the probe 11 are not necessarily matched. For example, the center frequency of the photoacoustic wave may be set at any position within the frequency band in the sensitivity of the probe 11 from a request for image quality or the like.

It is desirable that patterns of the excitation light generation condition as described above are stored in advance in a storage unit (not illustrated) inside the ultrasound unit 12 as a table and can be selected automatically as appropriate or by a user randomly according to the frequency characteristics of the probe 11, an observation target, an image depth (maximum depth in an image) or a focal depth (depth of an observation target) of photoacoustic image or the ultrasound image to be combined with the photoacoustic image, and/or the like.

It is possible to determine the frequency characteristics of the photoacoustic wave with a flow of setting a pulse waveform (excitation light generation condition) of the laser light L, generating a photoacoustic wave that can be regarded as a pseudo-differentiation of the pulse waveform of the laser light L, and performing frequency analysis (for example, Fourier transform) of a waveform of the generated photoacoustic wave to acquire the frequency characteristics of the photoacoustic wave. For this reason, it is possible to extract the excitation light generation condition that provides the desired frequency characteristics of the photoacoustic wave by repeating the above procedure while changing some parameters of the excitation light generation condition little by little.

In general, in a case where the pulse width of the laser light L is increased with respect to a photoacoustic wave generated by the pulse width and the pulse repetition period of certain laser light L, it is possible to generate the photoacoustic wave having frequency characteristics close to the original photoacoustic wave by shortening the pulse repetition period. Conversely, in a case where the pulse width of the laser light L is shortened with respect to a photoacoustic wave generated by the pulse width and the pulse repetition period of certain laser light L, it is possible to generate the photoacoustic wave having frequency characteristics close to the original photoacoustic wave by lengthening the pulse repetition period.

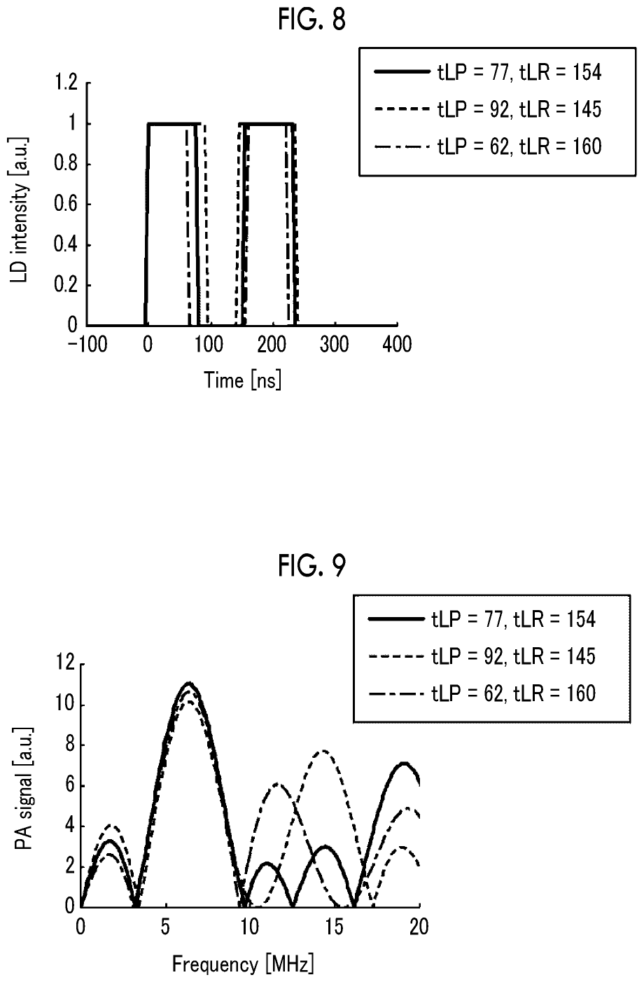

For example, FIG. 8 is a graph illustrating waveforms of the excitation light, and FIG. 9 is a graph illustrating spectra of the photoacoustic waves. As illustrated in FIGS. 8 and 9, in a case where the number of pulses of the laser light L is two, it is possible to generate a photoacoustic wave having the center frequency near 6.5 MHz (megahertz) in a case where the pulse width t_{LP} of laser light L is set to 77 ns (nanosecond) and the pulse repetition period t_{LR} of laser light L is set to 154 ns (nanosecond). However, it is possible to generate a photoacoustic wave having the center frequency near 6.5 MHz (megahertz) also in a case where the pulse width t_{LP} of the laser light L is set to 92 ns (nanosecond) and the pulse repetition period t_{LR} of the laser light L is set to 145 ns (nanosecond), and in a case where the pulse width t_{LP} of the laser light L is set to 62 ns (nanosecond) and the pulse repetition period t_{LR} of the laser light L is set to 160 ns (nanosecond).

With this method, it is possible to change the frequency characteristics of the generated photoacoustic wave even in a case where there is a limit to a ratio of time during which pulsed light is emitted while the laser unit 13 (light source) is driven (in general, a laser diode is often limited to an upper limit of 0.1%. In addition, there is a case where a total light emission amount is required to be reduced from the viewpoint of laser safety), or in a case where the pulse width is restricted (in a case where only a predetermined value can be selected for the pulse width of the laser light L such as in a case where there is a restriction of a circuit or the like, or in a case of a flash lamp type light source).

For example, in a case where a pulse repetition frequency (PRF) of the photoacoustic wave is high and it is desired to reduce the ratio of time during which the pulsed light is emitted during driving in one waveform acquisition, the excitation light generation condition is selected in which the pulse width t_{LP} of the laser light L is set to 62 ns (nanosecond) and the pulse repetition period t_{LR} of the laser light L is set to 160 ns (nanosecond). Therefore, it is possible to generate the photoacoustic wave having the center frequency near 6.5 MHz (megahertz) while the ratio of time during which the pulsed light is emitted during driving is reduced as compared with the case of t_{LP}=t_{LR}/2. In addition, in a case where there is a restriction that the pulse width t_{LP} of the laser light L is 92 ns (nanosecond) (the center frequency is 5.4 MHz at L_{LP}=t_{LR}/2), it is possible to generate the photoacoustic wave having the center frequency near 6.5 MHz (megahertz) in a case where the pulse repetition period t_{LR} of the laser light L is set to 145 ns (nanosecond).

In a case where a laser diode (LD) or a light emitting diode (LED) is used as the laser unit 13 (light source), a method capable of generating a photoacoustic wave having the same degree of intensity as a long light emission time in a short light emission time (for example, selection of the excitation light generation condition with the pulse width t_{LP}=62 ns (nanosecond) of the laser light L and the pulse repetition period t_{LR}=160 ns (nanosecond) of the laser light L in a case where a photoacoustic wave having the center frequency near 6.5 MHz (megahertz) is desired to be generated) is desirable in terms of low power consumption since a light emission time of the laser light L is proportional to power consumption. In addition, it is possible to expect a longer lifetime by shortening the light emission time since the lifetime of the light emitting device is considered to be caused by a total light emission time.

Next, a photoacoustic image generation apparatus 10 according to a second embodiment of the invention will be described. The photoacoustic image generation apparatus 10 according to the embodiment is different only in the control method of the laser unit 13 (light source) in the control unit 30 as compared with the photoacoustic image generation apparatus 10 according to the first embodiment and other configurations are the same. Therefore, a description of the same part is omitted.

In the embodiment, the control unit 30 performs control with respect to the laser unit 13 (light source) at the time of acquiring the photoacoustic image to adjust the excitation light generation condition based on the pulse width, the number of a plurality of pulses, and the pulse repetition period of the laser light L (excitation light) generated by the laser unit 13 such that the frequency characteristics of the photoacoustic wave immediately before the probe 11 are close to the reception frequency characteristics of the probe 11 (acoustic wave detection means) based on the reception frequency characteristics of the probe 11 (acoustic wave detection means) and a depth of an observation target. Here, a center frequency is considered as the frequency characteristics, but other frequency characteristics such as a peak frequency may be used.

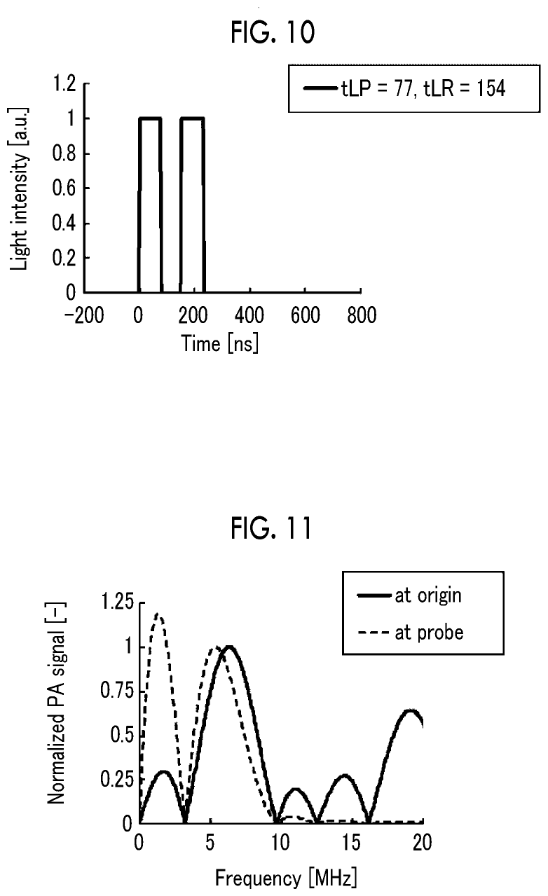

For example, a case is considered in which the center frequency in the sensitivity of the probe 11 is 6.5 MHz (megahertz) and the depth of a main observation target is 4 cm (centimeter). FIG. 10 is a graph illustrating a waveform of the excitation light, and FIG. 11 is a graph illustrating spectra of the photoacoustic waves. As illustrated in FIG. 10, the excitation light generation condition (the pulse width t_{LP}=77 ns (nanosecond) of the laser light L (excitation light) and the pulse repetition period t_{LR}=154 ns (nanosecond) of the laser light L) is set such that the center frequency of a photoacoustic wave generated in the subject M is 6.5 MHz (megahertz) in the first embodiment. However, the generated photoacoustic wave attenuates in the subject M (in particular, high-frequency components attenuate more). Therefore, the center frequency of the photoacoustic wave immediately before the probe 11 changes from 6.5 MHz (megahertz) to a lower center frequency as illustrated in FIG. 11.

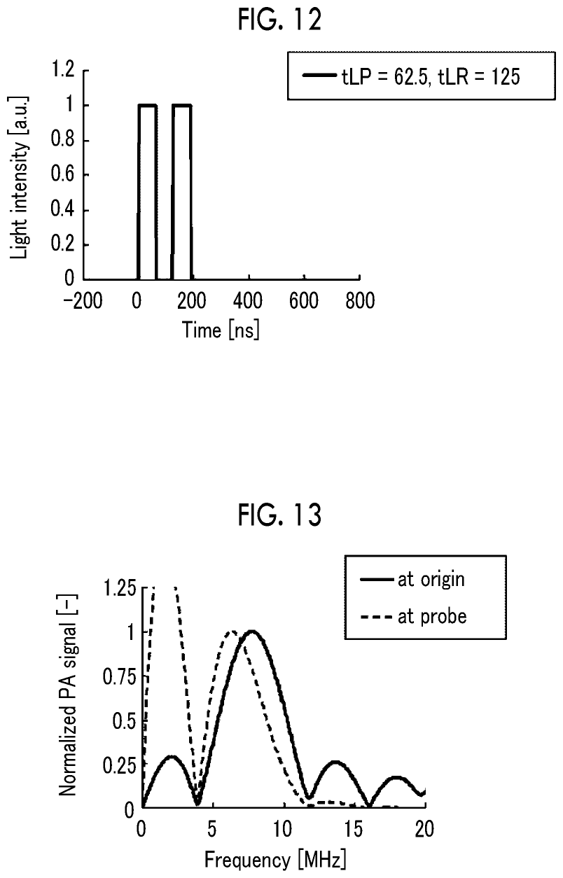

For this reason, in consideration of the attenuation in the subject M, the excitation light generation condition is set such that the center frequency of the photoacoustic wave immediately before the probe 11 is close to the center frequency in the sensitivity of the probe 11 in the embodiment. FIG. 12 is a graph illustrating a waveform of the excitation light, and FIG. 13 is a graph illustrating spectra of the photoacoustic waves. For example, in a case where the number of pulses of the laser light L is two, the pulse width t_{LP} of the laser light L is set to 62.5 ns (nanosecond) and the pulse repetition period t_{LR} of the laser light L is set to 125 ns (nanosecond) as illustrated in FIG. 12, it is possible to set the center frequency of the photoacoustic wave immediately before the probe 11 to 6.5 MHz (megahertz) as illustrated in FIG. 13. Accordingly, it is possible to enhance the reception efficiency of the photoacoustic wave in the probe 11 as compared with the first embodiment.

It is desirable that a plurality of patterns (for example, for a case where the main observation target is located in a shallow place, for a case where the main observation target is located in an intermediate place, and for a case where the main observation target is located in a deep place) of the excitation light generation condition at which the center frequency of the photoacoustic wave immediately before the probe 11 and the center frequency in the sensitivity of the probe 11 are close to each other are stored in advance in the ultrasound unit 12 as a table for each type of the probe 11 and can be selected by a user.

In this case, it is desirable that a detection condition of the photoacoustic wave is also optimized according to each mode. The excitation light generation conditions may be automatically switched according to an image depth (maximum depth in an image) or a focal depth (depth of an observation target) of the photoacoustic image or the ultrasound image to be combined with the photoacoustic image.

The number of pulses of the laser light L is not limited to two and may be three or more.

For example, in a case where the number of pulses of the laser light L increases, an object position in the photoacoustic image changes due to a change in the excitation light generation condition such as the object position in the photoacoustic image being shifted in a deep direction. For this reason, it is desirable that the photoacoustic image generation unit 24 corrects the object position in the photoacoustic image according to the excitation light generation condition.

The center frequency of the photoacoustic wave immediately before the probe 11 and the center frequency in the sensitivity of the probe 11 are not necessarily matched. For example, the center frequency of the photoacoustic wave may be set at any position within the frequency band in the sensitivity of the probe 11 from a request for image quality or the like.

Next, a photoacoustic image generation apparatus 10 according to a third embodiment of the invention will be described. The photoacoustic image generation apparatus 10 according to the embodiment is different only in the control method of the laser unit 13 (light source) in the control unit 30 as compared with the photoacoustic image generation apparatus 10 according to the first embodiment and other configurations are the same. Therefore, a description of the same part is omitted.

In the embodiment, the control unit 30 performs control with respect to the laser unit 13 (light source) at the time of acquiring the photoacoustic image to select an optimum setting from a plurality of settings having different center frequencies of photoacoustic waves generated in the subject M for the excitation light generation condition based on the pulse width, the number of plurality of pulses, and the pulse repetition period of the laser light L (excitation light) generated by the laser unit 13 so as to obtain appropriate reception characteristics such as resolution priority or sensitivity priority based on the reception frequency characteristics of the probe 11 (acoustic wave detection means) and a depth of an observation target. Here, a center frequency is considered as the frequency characteristics, but other frequency characteristics such as a peak frequency may be used.

Specifically, the photoacoustic wave generated in the subject M attenuates in the subject M (in particular, high-frequency components attenuate more) and reaches the probe 11. For this reason, in a case where the observation target is at a deep position, the excitation light generation condition is adjusted such that a photoacoustic wave in a low frequency side with a small attenuation rate per unit length is generated. In a case where the observation target is at a shallow position, an influence of attenuation is small. Therefore, the excitation light generation condition is adjusted such that a photoacoustic wave in a high frequency side with a high resolution is generated. Accordingly, a more desirable photoacoustic image is obtained at each depth.

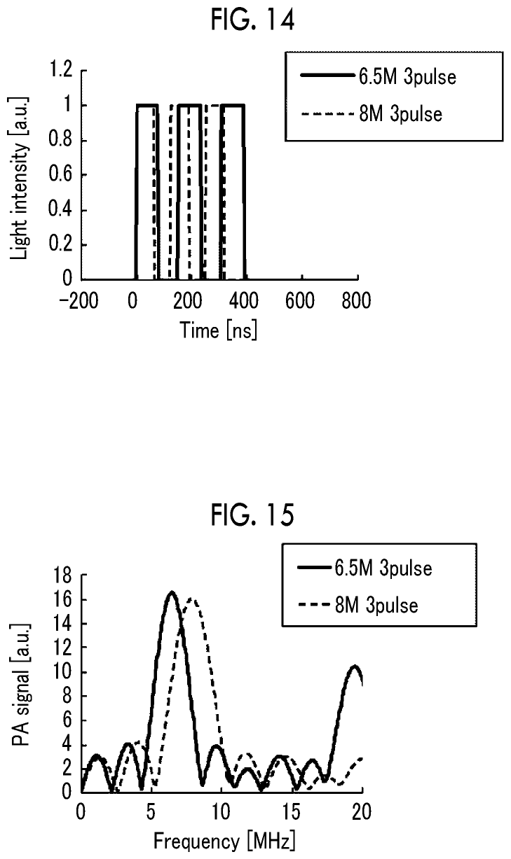

For example, a case is considered in which the number of pulses is three and the center frequencies of photoacoustic waves generated in the subject M are 6.5 MHz (megahertz) and 8 MHz (megahertz), respectively. FIG. 14 is a graph illustrating waveforms of the excitation light, and FIG. 15 is a graph illustrating spectra of the photoacoustic waves. As illustrated in FIG. 14, the excitation light generation condition such that the center frequency of the photoacoustic wave generated in the subject M is 6.5 MHz (megahertz) is the pulse width L_{LP}=77 ns (nanosecond) of the laser light L (excitation light) and the pulse repetition period t_{LR}=154 ns (nanosecond) of the laser light L. The excitation light generation condition such that the center frequency of the photoacoustic wave generated in the subject M is 8 MHz (megahertz) is the pulse width t_{LP}=62.5 ns (nanosecond) of the laser light L (excitation light) and the pulse repetition period t_{LR}=125 ns (nanosecond) of the laser light L. Further, the spectra of the photoacoustic waves generated under these conditions are as illustrated in FIG. 15.

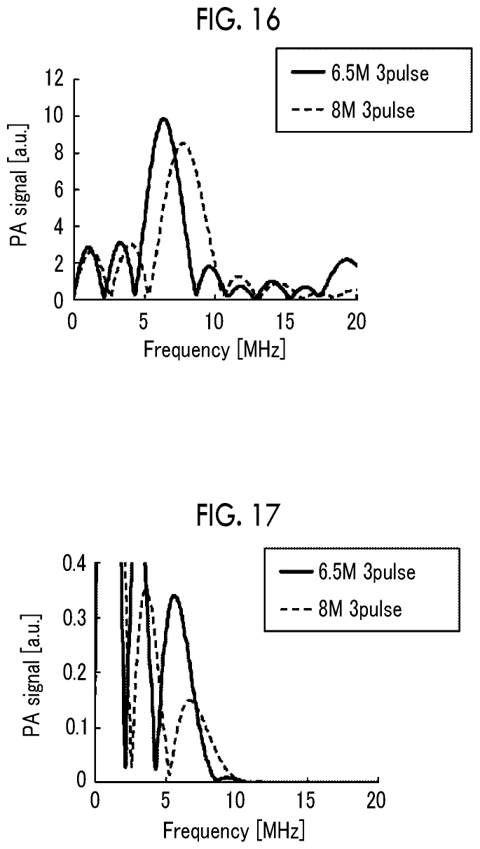

As illustrated in FIG. 16 which is a graph illustrating spectra of the photoacoustic waves, in a case where the depth of the observation target is shallow (for example, 1 cm (centimeter)), a difference in intensity between two kinds of photoacoustic waves is small since the influence of attenuation in the subject M is small. In such a case, a high frequency waveform may be selected with an emphasis on the resolution. The main component of the high frequency photoacoustic wave is in a receivable frequency band (about 3.2 to 9.8 MHz (megahertz)) of the probe having the center frequency of 6.5 MHz (megahertz).

As illustrated in FIG. 17 which is a graph illustrating spectra of the photoacoustic waves, in a case where the observation target is deep (for example, 8 cm (centimeter)), a difference in intensity between two kinds of photoacoustic waves is large since the influence of attenuation in the subject M is large. In such a case, a low frequency waveform may be selected with an emphasis on sensitivity. The main component of the low frequency photoacoustic wave is in the receivable frequency band (about 3.2 to 9.8 MHz (megahertz)) of the probe having the center frequency of 6.5 MHz (megahertz).

In this manner, it is possible to acquire a suitable photoacoustic image for each depth of the observation target by adjusting the excitation light generation condition based on the depth of the observation target.

It is desirable that a plurality of patterns (for example, for a case where the main observation target is located in a shallow place, for a case where the main observation target is located in an intermediate place, and for a case where the main observation target is located in a deep place) of the excitation light generation condition at which the center frequency of the photoacoustic wave immediately before the probe 11 and the center frequency in the sensitivity of the probe 11 are close to each other are stored in advance in the ultrasound unit 12 as a table for each type of the probe 11 and can be selected by a user.

In this case, it is desirable that a detection condition of the photoacoustic wave is also optimized according to each mode. The excitation light generation conditions may be automatically switched according to an image depth (maximum depth in an image) or a focal depth (depth of an observation target) of the photoacoustic image or the ultrasound image to be combined with the photoacoustic image.

The number of pulses of the laser light L is not limited to three and may be two or more.

For example, in a case where the number of pulses of the laser light L increases, an object position in the photoacoustic image changes due to a change in the excitation light generation condition such as the object position in the photoacoustic image being shifted in a deep direction. For this reason, it is desirable that the photoacoustic image generation unit 24 corrects the object position in the photoacoustic image according to the excitation light generation condition.

The center frequency of the photoacoustic wave immediately before the probe 11 and the center frequency in the sensitivity of the probe 11 are not necessarily matched. For example, the center frequency of the photoacoustic wave may be set at any position within the frequency band in the sensitivity of the probe 11 from a request for image quality or the like.

The invention has been described above based on the preferred embodiments. However, the photoacoustic image generation apparatus according to the embodiment of the invention is not limited only to the above-described embodiments. Various modifications and changes of the configurations according to the above-described embodiments are also included in the scope of the invention.

What is claimed is:

1. A photoacoustic image generation apparatus comprising:
a processor configured to generate a photoacoustic image based on a signal obtained by detecting a photoacoustic wave, using an acoustic wave detection means, generated from an inside of a subject by receiving excitation light emitted from a light source toward the subject,
wherein the processor is further configured to perform control to adjust an excitation light generation condition based on a pulse width, the number of a plurality of pulses, and a pulse repetition period of the excitation light generated in the light source based on reception frequency characteristics of the acoustic wave detection means with respect to the light source.

2. The photoacoustic image generation apparatus according to claim 1,
wherein the processor performs control to adjust the excitation light generation condition such that frequency characteristics of the photoacoustic wave detected by the acoustic wave detection means are close to the reception frequency characteristics of the acoustic wave detection means.

3. The photoacoustic image generation apparatus according to claim 1,
wherein the processor stores a plurality of excitation light generation conditions having different frequency characteristics of photoacoustic waves generated in the subject and controls the light source based on the excitation light generation condition selected from among the plurality of stored excitation light generation conditions.

4. The photoacoustic image generation apparatus according to claim 3,
wherein the processor stores a plurality of excitation light generation conditions for each type having different reception frequency characteristics of the acoustic wave detection means and controls the light source based on the excitation light generation condition selected by a user from among the plurality of stored excitation light generation conditions.

5. The photoacoustic image generation apparatus according to claim 1,
wherein the processor adjusts the excitation light generation condition based on an image depth of the photoacoustic image.

6. The photoacoustic image generation apparatus according to claim 1,
wherein the processor adjusts the excitation light generation condition based on a focal depth of the photoacoustic image.

7. The photoacoustic image generation apparatus according to claim 1,
wherein the processor performs correction processing on the photoacoustic image based on the excitation light generation condition.

8. An image acquisition method in a photoacoustic image generation apparatus comprising a processor configured to generate a photoacoustic image based on a signal obtained by detecting a photoacoustic wave, using an acoustic wave detection means, generated from an inside of a subject by receiving excitation light emitted from a light source toward the subject, the method comprising:
performing control to adjust an excitation light generation condition based on a pulse width, the number of a plurality of pulses, and a pulse repetition period of the excitation light generated in the light source based on reception frequency characteristics of the acoustic wave detection means with respect to the light source.

9. The image acquisition method according to claim 8, further comprising:
performing control to adjust the excitation light generation condition such that frequency characteristics of the photoacoustic wave detected by the acoustic wave detection means are close to the reception frequency characteristics of the acoustic wave detection means.

10. The image acquisition method according to claim 8, further comprising:
storing a plurality of excitation light generation conditions having different frequency characteristics of photoacoustic waves generated in the subject and controlling the light source based on the excitation light generation condition selected from among the plurality of stored excitation light generation conditions.

11. The image acquisition method according to claim 10, further comprising:
storing a plurality of excitation light generation conditions for each type having different reception frequency characteristics of the acoustic wave detection means and controlling the light source based on the excitation light generation condition selected by a user from among the plurality of stored excitation light generation conditions.

12. The image acquisition method according to claim 8, further comprising:
adjusting the excitation light generation condition based on an image depth of the photoacoustic image.

13. The image acquisition method according to claim 8, further comprising:
adjusting the excitation light generation condition based on a focal depth of the photoacoustic image.

14. The image acquisition method according to claim 8, further comprising:
performing correction processing on the photoacoustic image based on the excitation light generation condition.
